(19) United States
(12) Patent Application Publication
(10) Pub. No.: US 2011/0137040 A1
(43) Pub. Date: Jun. 9, 2011

(54) SYNTHESIS OF 3,4-DIARYL-4,5-DIHYDRO-(H)-PYRAZOLE-1-CARBOXAMIDINE DERIVATIVES

(76) Inventors: Josephus H.M. Lange, Weesp (NL); Hans J. Sanders, Weesp (NL); Jeroen Van Rheenen, Weesp (NL)

(21) Appl. No.: 13/056,861

(22) PCT Filed: Jul. 30, 2009
(86) PCT No.: PCT/EP2009/059844

§ 371 Date: Jan. 31, 2011

Related U.S. Application Data
(60) Provisional application No. 61/085,475, filed on Aug. 1, 2008.
(60) Provisional application No. 61/085,475, filed on Aug. 1, 2008.

(51) Int. Cl. C07D 231/06; C07D 401/12

(52) U.S. Cl. 546/211; 548/379.4

(57) ABSTRACT

The invention relates to a novel chemical route to 3,4-diaryl-4,5-dihydro-(1H)-pyrazole-1-carbox-amidine derivatives, known as potent cannabinoid-CB₁ receptor antagonists, and to novel intermediates of these compounds. The synthetic route produced considerably higher yields than those reported, without the use of corrosive reagents. The process concerns the preparation of a compound of formula (I):

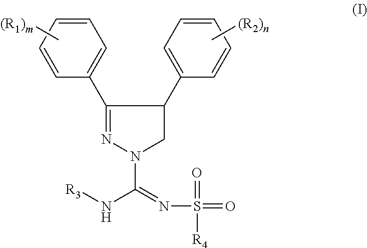

wherein the symbols have the meanings given in the description.

This invention relates to organic chemistry, in particular to processes for the preparation of 3,4-diaryl-4,5-dihydro-(1H)-pyrazole-1-carboxamidine derivatives, known as potent cannabinoid-CB₁ receptor antagonists. The invention also relates to novel intermediates of these compounds.

### BACKGROUND

Compounds A and B are 3,4-diaryl-4,5-dihydro-1H-pyrazole-1-carboxamidine derivatives representative for the cannabinoid-CB₁ receptor antagonists disclosed in WO 01/70700 and WO 03/026648.

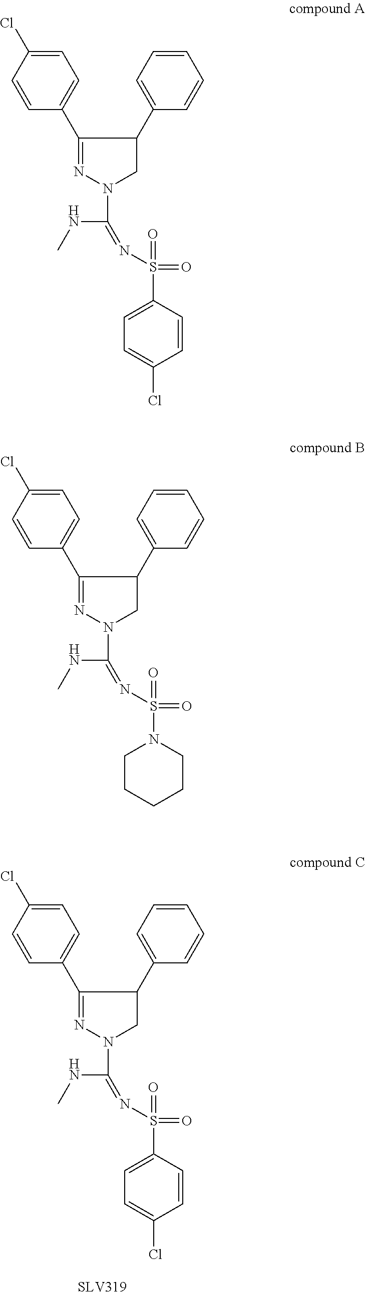

Chiral chromatographic separation of racemates A and B yielded the optically pure compound C (SLV319, (ibipinabant) disclosed in WO 02/076949), and the corresponding (4S)-enantiomer of compound B, respectively. The synthetic routes disclosed in the patents quoted above have reasonable yields, but they are not ideally suited for synthesis on the scale required for drugs in clinical development, let alone on the scale required for marketed drugs. The yield of compound A from its key intermediate 3-(4-chlorophenyl)-4-phenyl-4,5-dihydro-1H-pyrazole was reported to be 60% (Lange, J. H. M., et al, J. Med. Chem., 2004, 47, 627), that of compound B 45% (WO 03/026648). In the known synthetic route to compound A, the corrosive chlorinating reactant PCl₅ is used at reflux temperature in chlorobenzene. At elevated temperatures PCl₅ is known to slowly decompose into PCl₃ and highly toxic chlorine gas (Cl₂). Large scale use of such compounds creates insurmountable safety hazards.

The objective of the present invention was to develop a novel synthetic route to 3,4-diaryl-4,5-dihydro-(1H)-pyrazole-1-carboxamidine derivatives, with higher yields than the known routes, and avoiding the use of corrosive reagents.

### DISCLOSURE

It was found that—without the use of corrosive reagents—a novel synthetic route to 3,4-diaryl-4,5-dihydro-(1H)-pyrazole-1-carboxamidine derivatives of general formula (I) produced substantially higher yields than those reported. That for compound A for instance, was 77%, that for compound B 73% from the key intermediate 3-(4-chlorophenyl)-4-phenyl-4,5-dihydro-1H-pyrazole. These are significantly higher than those reported (60% and 45% for A and B respectively). The invention relates to a process for the preparation of a compound of formula (I):

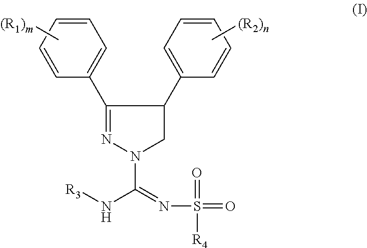

wherein:
R₁ and R₂ independently are chosen from (C₁₋₃)-alkyl or (C₁₋₃)-alkoxy, hydroxy, halogen, trifluoromethyl, trifluoromethoxy and cyano,
m and n independently are 0, 1 or 2,
R₃ is branched or linear (C₁₋₈)-alkyl or (C₃₋₈)-cycloalkyl,
R₄ is chosen from phenyl, thienyl or pyridyl, which groups are unsubstituted or substituted with 1 or 2 substituents, which can be the same or different, chosen from (C₁₋₃)-alkyl or (C₁₋₃)-alkoxy, hydroxy, halogen, trifluoromethyl, trifluoromethoxy and cyano, or
   R₄ represents a monocyclic or bicyclic (C₅₋₁₀)-alkyl or (C₅₋₁₀)-alkenyl group, or a monocyclic or bicyclic hetero-(C₅₋₁₀)-alkyl or hetero-(C₅₋₁₀)-alkenyl group containing one or two ring heteroatoms or ring heteroatom-containing moieties chosen from N, O, S or SO₂, and which R₄ group is unsubstituted or substituted with a substituent chosen from hydroxy or (C₁₋₃)-alkyl or R₄ represents a 4,4-difluoropiperidin-1-yl, 4-fluoropiperidin-1-yl or 4-(trifluoro-methyl)piperidin-1-yl group.

The invention also relates to a process for the preparation of a compound of formula (I) wherein R₁ and R₂ independently are chosen from (C₁₋₃)-alkyl, trifluoromethyl or halogen; m and n independently are 0 or 1; R₃ is branched or linear (C₁₋₃)-alkyl; R₄ represents phenyl, unsubstituted or substituted with 1 substituent chosen from (C₁₋₃)-alkyl, trifluoromethyl or halogen, or R₄ represents a monocyclic hetero-(C₅₋₁₀)-alkyl group, which contains one or two ring heteroatoms chosen from N, O and S or R₄ represents a 4,4-difluoropiperidin-1-yl, 4-fluoropiperidin-1-yl or 4-(trifluoromethyl)piperidin-1-yl group.

Another embodiment relates to a process for the preparation of a compound of formula (I) wherein R₁ and R₂ are halogen; m and n independently are 0 or 1; R₃ is methyl; R₄ represents phenyl, unsubstituted or substituted with 1 halogen atom, or R₄ represents a piperidin-1-yl or 4,4-difluoropiperidin-1-yl group.

A further embodiment provides a process for the preparation of a compound of formula (I) wherein R₁ is 4-Cl; m is 1 and n is 0; R₃ is methyl, and R₄ is chosen from 4-chlorophenyl, piperidin-1-yl and 4,4-difluoropiperidin-1-yl.

Specific embodiments relate to processes for the preparation of compounds having formulae:

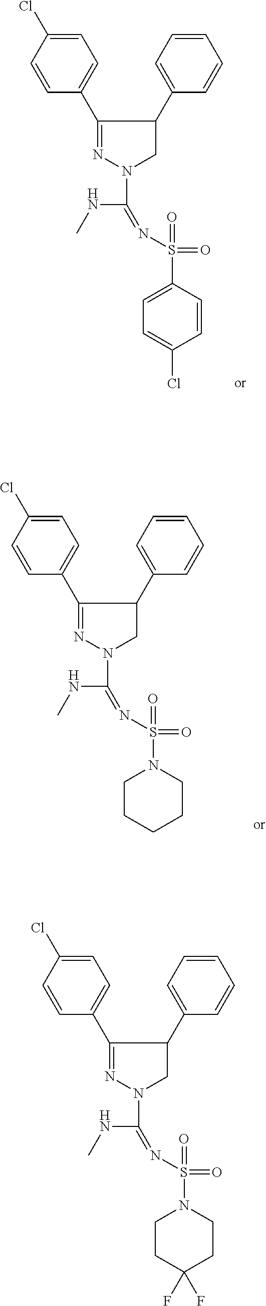

Further embodiments provide one or more compounds of formula (III) or (IIIa):

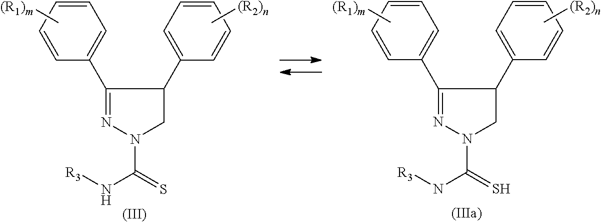

wherein R₃ is branched or linear (C₁₋₈)-alkyl, and the other symbols have the meanings given above, as well as tautomers, stereoisomers, N-oxides, and salts of any of the foregoing. Such compounds are useful in the synthesis of compounds of formula (I).

Further embodiments provide one or more compounds of formula (IV)

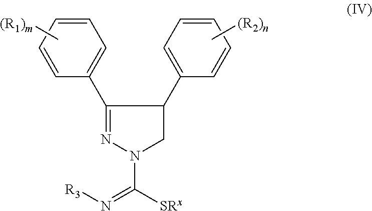

wherein R^{x} represents a linear (C₁₋₈)alkyl group, and the symbols have the meanings given above, as well as tautomers, stereoisomers, N-oxides, and salts of any of the foregoing. Such compounds are useful in the synthesis of compounds of formula (I).

Isolation and purification of the compounds and intermediates described herein can be affected, if desired, by any suitable separation or purification procedure, for example, filtration, extraction, crystallization, column chromatography, thin-layer chromatography, thick-layer chromatography, preparative low or high-pressure liquid chromatography, or a combination of these procedures. Specific illustrations of suitable separation and isolation procedures can be taken from the preparations and examples. However, other equivalent separation or isolation procedures could, of course, also be used.
The compounds of the present invention may contain one or more chiral centers and can thus occur as racemates and racemic mixtures, single enantiomers, diastereomeric mixtures and individual diastereomers. All compounds of the present invention contain a chiral center at the C₄ atom of their 4,5-dihydro-1H-pyrazole moiety.
Depending on the nature of the various substituents, the molecule can have additional asymmetric centers. Each such asymmetric center will independently produce two optical isomers. All of the possible optical isomers, enantiomers and diastereomers, in mixtures and as pure or partially purified compounds, belong to this invention. The present invention comprehends all such isomeric forms of these compounds. Formulae (III), (IIIa) and (IV) show the structure of the class of compounds without preferred stereochemistry. The independent syntheses of these optical isomers, or their chromatographic separations, may be achieved as known in the art by appropriate modification of the methodology disclosed therein. Their absolute stereochemistry may be determined by the X-ray crystallography of crystalline products or crystalline intermediates, which are derivatized, if necessary, with a reagent containing a chiral center of known absolute configuration. Racemic mixtures of the compounds can be separated into the individual enantiomers by methods well-known in the art, such as the coupling of a racemic mixture of compounds to an enantiomerically pure compound to form a diastereomeric mixture, followed by separation of the individual diastereomers by standard methods, such as fractional crystallization or chromatography. The coupling often consists of the formation of salts using an enantiomerically pure acid or base, for example (−)-di-p-toluoyl-D-tartaric acid or (+)-di-p-toluoyl-L-tartaric acid. The diasteromeric derivatives may then be converted to the pure enantiomers by cleavage of the added chiral residue. The racemic mixture of the compounds can also be separated directly by chromatographic methods utilizing chiral stationary phases, well-known in the art. Alternatively, any enantiomer of a compound may be obtained by stereoselective synthesis using optically pure starting materials or reagents of known configuration by methods well-known in the art.
Cis and trans isomers of compounds of formulae (III), (IIIa) and (IV), or salts thereof, also belong to the invention, and this applies to their tautomers, too.
The synthetic strategy in this novel route is essentially different from the known routes since in those the R₃—NH moiety in the compound of general formula (I) was introduced by a nucleophilic displacement of a leaving group—such as a chloro atom or a methylsulfanyl group—in the last step of the reaction sequence. In the novel route the R₃NH group is introduced at a much earlier stage in the process as an electrophile (R₃-isothiocyanate) via reaction with the nucleophilic pyrazoline building block (II). In the novel route the R₄SO₂N moiety in the compound of general formula (I) is introduced in the last step of the reaction sequence, whereas in all prior art routes this particular moiety was introduced at an earlier stage in the process.

### DEFINITIONS

General terms used in the description of compounds herein disclosed bear their usual meanings. The term alkyl as used herein denotes a univalent saturated, branched or linear, hydrocarbon chain. Unless otherwise stated, such chains can contain from 1 to 18 carbon atoms. Representative of such alkyl groups are methyl, ethyl, propyl, isopropyl, butyl, isobutyl, sec-butyl, tert-butyl, pentyl, isopentyl, neopentyl, hexyl, isohexyl, heptyl, octyl, nonyl, decyl, undecyl, dodecyl, tridecyl, tetradecyl, pentadecyl, hexadecyl, heptadecyl, octadecyl, etc. The same carbon content applies to the parent term ‘alkane’, and to derivative terms like ‘alkoxy’. The carbon content of various hydrocarbon containing moieties is indicated by a prefix designating the minimum and maximum number of carbon atoms, i.e., the prefix (C_{x-y})—defines the number of carbon atoms present from the integer “x” to the integer “y” inclusive. ‘(C₁₋₃)-alkyl’ for example, includes methyl, ethyl, n-propyl or isopropyl, and ‘(C₁₋₄)-alkyl’ includes ‘methyl, ethyl, n-propyl, isopropyl, n-butyl, sec-butyl, isobutyl or tert-butyl’. The term ‘alkenyl’ denotes linear or branched hydrocarbon radicals having one or more carbon-carbon double bonds, such as vinyl, allyl, butenyl, etc., and for example represents (C₂₋₄)alkenyl.

‘Halo’ or ‘Halogen’ refers to chloro, fluoro, bromo or iodo; ‘hetero’ as in ‘heteroalkyl, heteroaromatic’, etc. includes containing one or more N, O or S atoms. ‘heteroalkyl’ includes alkyl groups with heteroatoms in any position, thus including N-bound O-bound or S-bound alkyl groups.

The term “substituted” means that the specified group or moiety bears one or more substituents. Where any group may carry multiple substituents, and a variety of possible substituents can be provided, the substituents are independently selected, and need not to be the same. The term “unsubstituted” means that the specified group bears no substituents. With reference to substituents, the term “independently” means that when more than one of such substituents are possible, they may be the same or different from each other.

‘C₃₋₈-cycloalkyl’ includes cyclopropyl, cyclobutyl, cyclopentyl, cyclohexyl, cyclopheptyl or cyclooctyl; ‘C₅₋₁₀ bicycloalkyl group’ refers to carbo-bicyclic ring systems including bicyclo[2.2.1]heptanyl, bicyclo[3.3.0]octanyl or the bicyclo[3.1.1]heptanyl group;

The term “amino” as used herein alone, or as part of another group, refers to a nitrogen atom being either terminal, or a linker between two other groups, wherein the group may be a primary, secondary or tertiary (two hydrogen atoms bonded to the nitrogen atom, one hydrogen atom bonded to the nitrogen atom and no hydrogen atoms bonded to the nitrogen atom, respectively) amine. The terms “sulfinyl” and “sulfonyl” as used herein as part of another group respectively refer to an —SO— or an —SO₂— group.

To provide a more concise description, the terms ‘compound’ or ‘compounds’ include tautomers, stereoisomers, N-oxides, isotopically-labelled analogues, or pharmacologically acceptable salts, also when not explicitly mentioned.

As used herein, the term “leaving group” (L) shall mean a charged or uncharged atom or group leaving during a substitution or displacement reaction. The term refers to groups readily displaceable by a nucleophile, such as an amine, a thiol or an alcohol nucleophile. Such leaving groups are well known in the art. Examples include, but are not limited to, N-hydroxysuccinimide, N-hydroxybenzotriazole, halides (Br, Cl, I), triflates, mesylates, tosylates, and the like. (For more information on the leaving group concept, see: Michael B. Smith and Jerry March, Advanced organic chemistry, reactions, mechanisms and structure, fifth edition, John Wiley & Sons, Inc., New York, 2001, p. 275).

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to experimental or measurement conditions for such given value.

Throughout the description and the claims of this specification the word “comprise” and variations of the word, such as “comprising” and “comprises” is not intended to exclude other additives, components, integers or steps.

### EXAMPLE 1

### Analytical Methods

¹H NMR spectra were recorded on a Varian UN400 instrument (400 MHz) or a Bruker Avance DRX600 instrument (600 MHz) using DMSO-d₆ or CDCl₃ as solvents with tetramethylsilane as an internal standard. Chemical shifts are given in ppm (δ scale) downfield from tetramethylsilane. Coupling constants (J) are expressed in Hz. Flash chromatography was performed using silica gel 60 (0.040-0.063 mm, Merck). Column chromatography was performed using silica gel 60 (0.063-0.200 mm, Merck) or alumina (act III). Sepacore chromatographic separations were carried out using Supelco equipment, VersaFLASH™ columns, VersaPak™ silica cartridges, Büchi UV monitor C-630, Büchi Pump module C-605, Bûchi fraction collector C-660 and Büchi pump manager C-615. Melting points were recorded on a Büchi B-545 melting point apparatus or determined by DSC (differential scanning calorimetry) methods.

### EXAMPLE 2

### General Aspects of Syntheses

3,4-diaryl-4,5-dihydro-1H-pyrazole-1-carboxamidine derivatives of formula (II) can be obtained via known methods, as described in WO01/70700, WO 03/026648, Lange, J. H. M. et al., J. Med. Chem. 2004, 47, 627. The novel synthetic route is given in the scheme below:

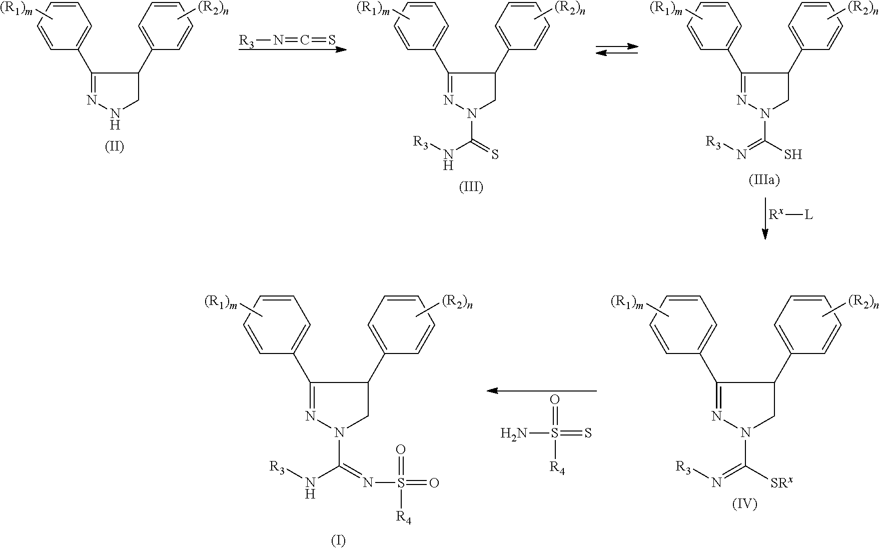

3,4-Diaryl-4,5-dihydro-(1H)-pyrazoles of formula (II) can be prepared as described by Grosscurt, et al. (J. Agric. Food Chem. 1979, 27, 406), and can be reacted with an alkylisothiocyanate, or a cycloalkylisothiocyanate, in a (C₁₋₈)-alcohol such as absolute ethanol, to give a 3,4-diaryl-N-alkyl-4,5-dihydro-(1H)-pyrazole-1-carbothioamide or 3,4-diaryl-N-cycloalkyl-4,5-dihydro-(1H)-pyrazole-1-carbothioamide of formula (III). In a (C₁₋₈)-alcohol such as methanol, the latter can be reacted with an alkylating reagent of general formula R^{x}-L, wherein R^{x} represents a linear (C₁₋₈)alkyl group and L represents a ‘leaving group’, preferably chosen from Br, Cl or I, to give a compound of formula (IV). In an inert organic solvent such as acetonitrile, a compound of formula (IV) can be reacted with a sulfonamide derivative of formula R₄SO₂NH₂, resulting in a compound of formula (I). A skilled person will notice that the group —SR^{x} acts as a leaving group in this particular reaction. In the scheme above, R₁, R₂, R₃, R₄, m and n have the meanings as given above. Compounds (IIIa) are tautomers of compounds (III), and as such part of the invention. Compounds of formulae (III), (IIIa) and (IV) are new.

Salts may be obtained using standard procedures well known in the art, for example by mixing a compound of the present invention with a suitable acid, for instance an inorganic acid such as hydrochloric acid, or with an organic acid such as fumaric acid.

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

According to these procedures the compounds described below have been prepared. They are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only.

### EXAMPLE 3

### Synthesis and Spectral Data of Intermediates

3-(4-Chlorophenyl)-4-phenyl-4,5-dihydro-1H-pyrazole (formula (II) wherein m=1, R₁=4-Cl and n=0) was prepared according to the procedure described by Grosscurt, A. C. et al., (J. Agric. Food Chem. 1979, 27, 406).

### 3-(4-Chlorophenyl)-N-methyl-4-phenyl-4,5-dihydro-1H-pyrazole-1-carbothioamide

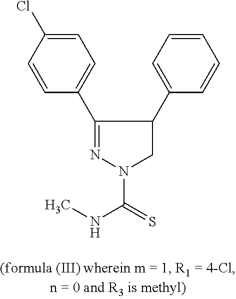

A mixture of 3-(4-chlorophenyl)-4-phenyl-4,5-dihydro-1H-pyrazole (30 g, 117 mmol), absolute ethanol (180 ml) and methylisothiocyanate (11.1 g, 152 mmol) was magnetically stirred under a nitrogen atmosphere at reflux temperature for 3 hours. The solid was filtered off and washed with ethanol (3×70 ml) and dried under vacuum to give a white solid (35 g, 90% yield). ¹H-NMR (400 MHz, CDCl₃): δ 3.25 (d, J=5 Hz, 3H), 4.33-4.45 (m, 1H), 4.63-4.73 (m, 2H), 7.12-7.18 (m, 2H), 7.22-7.36 (m, 5H), 7.44 (br s, 1H), 7.56 (d, J=8.7 Hz, 2H). Melting point: 181-183° C. ¹³C-NMR (100 MHz, CDCl₃): δ 31.5, 50.6, 58.6, 127.2 (2C), 127.8, 128.5 (2C), 128.85, 128.88 (2C), 129.4 (2C), 136.2, 139.6, 155.9, 177.0.

### Methyl 3-(4-chlorophenyl)-N-methyl-4-phenyl-4,5-dihydro-1H-pyrazole-1-carbothioimidate

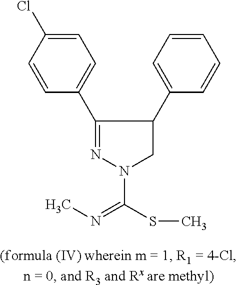

To a magnetically stirred solution of 3-(4-chlorophenyl)-N-methyl-4-phenyl-4,5-dihydropyrazole-1-carbothioamide (5 g, 15.2 mmol) in methanol (150 ml) was added iodomethane (20 ml, 322 mmol). The mixture was reacted at 40° C. (oilbath temperature) overnight under a nitrogen atmosphere. The solution was concentrated in vacuum with an oilbath temperature below 45° C. The residue was re-dissolved in dichloromethane (300 ml). The mixture was washed with saturated aqueous NaHCO₃ solution (70 ml) and brine (70 ml), dried over sodium sulfate, filtered and concentrated in vacuum to afford methyl 3-(4-chlorophenyl)-N-methyl-4-phenyl-4,5-dihydro-1H-pyrazole-1-carbothioimidate (5.2 g, 99% yield) as a yellow solid. ¹H-NMR (400 MHz, CDCl₃): δ 2.64 (s, 3H), 3.25 (s, 3H), 3.88 (dd, J=11 and 4.5 Hz, 1H), 4.37 (t, J=11 Hz, 1H), 4.56 (dd, J=11 and 4.5 Hz, 1H), 7.15-7.33 (m, 7H), 7.56 (d, J=8.7 Hz, 2H). ¹³C-NMR (100 MHz, CDCl₃): δ 16.7, 38.5, 49.8, 58.1, 127.2 (2C), 127.4, 127.7 (2C), 128.6 (2C), 129.1 (2C), 130.1, 134.7, 140.0, 152.5, 154.1. It should be noted that performance of this experiment under the same conditions with the exception of the used amount of methyliodide (10 molar equivalents instead of 21.2 molar equivalents) gave complete conversion to methyl 3-(4-chlorophenyl)-N-methyl-4-phenyl-4,5-dihydro-1H-pyrazole-1-carbothioimidate.

### EXAMPLE 4

### Syntheses of Specific Compounds

### 3-(4-Chlorophenyl)-N-methyl-4-phenyl-N′-(4-chlorophenylsulfonyl)-4,5-dihydro-1H-pyrazole-1-carboxamidine (Compound A, Structure Shown Above)

To a magnetically stirred solution of methyl 3-(4-chlorophenyl)-N-methyl-4-phenyl-4,5-dihydro-1H-pyrazole-1-carbothioimidate (4.00 g, 11.62 mmol) and 4-chlorobenzenesulfonamide (2.34 g, 12.20 mmol) in acetonitrile (90 ml) was heated at reflux temperature for 16 hours. The resulting mixture was evaporated in vacuum. The obtained crude residue was further purified by flash chromatography (silica gel, eluant gradient: petroleum ether/ethyl acetate=90/10=>80/20=>70/30=>60/40 (v/v/)) to afford compound A (4.93 gram, 87% yield) as a solid. ¹H-NMR (400 MHz, CDCl₃): δ 3.23 (d, J=5 Hz, 3H), 4.10 (dd, J=11 and 4.5 Hz, 1H), 4.53 (t, J=11 Hz, 1H), 4.64 (dd, J=11 and 4.5 Hz, 1H), 7.05-7.18 (m, 3H), 7.23-7.34 (m, 5H), 7.38 (br d, J˜8.5 Hz, 2H), 7.52 (br d, J˜8.5 Hz, 2H), 7.85 (br d, J˜8.5 Hz, 2H).

### 3-(4-Chlorophenyl)-N-methyl-4-phenyl-N′-(piperidin-1-ylsulfonyl)-4,5-dihydro-1H-pyrazole-1-carboxamidine (Compound B, Structure Shown Above)

A solution of methyl 3-(4-chlorophenyl)-N-methyl-4-phenyl-4,5-dihydro-1H-pyrazole-1-carbo-thioimidate (5.0 g, 14.5 mmol) and piperidine-1-sulfonamide (2.5 g, 15.23 mmol) in acetonitrile (110 ml) was stirred at 90° C. overnight. The yellow solution was evaporated in vacuum. Purification by column chromatography on alumina (Act. III) eluting with an heptane/ethyl acetate gradient from 3/1 to 1/1 gave compound B (5.5 g, 82% yield, 99% HPLC purity) as a white solid. Compound B crystallized in the test tubes from the column (heptane/EtOAc 2/1) as nice needles. ¹H-NMR (600 MHz; DMSO-d₅) δ 1.41-1.46 (m, 2H), 1.53-1.60 (m, 4H), 2.94-3.00 (m, 4H), 3.04 (br s, 3H), 4.07 (br d, J˜11 Hz, 1H), 4.51 (t, J˜11 Hz, 1H), 5.00 (dd, J˜11 and 4 Hz, 1H), 7.21-7.26 (m, 3H), 7.30-7.34 (m, 2H), 7.38 (d, J˜8 Hz, 2H), 7.74 (d, J˜8 Hz, 2H).

### N-[(4,4-difluoropiperidin-1-yl)sulfonyl]-N′-methyl-3-(4-chlorophenyl)-4-phenyl-4,5-dihydro-(1H)-pyrazole-1-carboxamidine (Compound D)

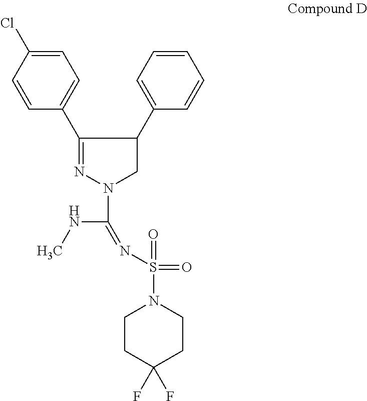

A solution of methyl 3-(4-chlorophenyl)-N-methyl-4-phenyl-4,5-dihydro-1H-pyrazole-1-carbo-thioimidate (4.0 g, 11.62 mmol) and 4,4-difluoropiperidine-1-sulfonamide (2.44 g, 12.2 mmol) in acetonitrile (110 ml) was stirred at 90° C. overnight under a nitrogen atmosphere. The reaction mixture was concentrated in vacuum. Purification by column chromatography on silica gel, eluting with a petroleum ether (40-65)/ethyl acetate gradient ranging from 9/1 to 8/2 to 7/3 to 6/4 (v/v) gave compound D (4.28 g, 71% yield) which was contaminated with some 4,4-difluoropiperidine-1-sulfonamide. Dissolution of the residue in dichloromethane and repeated washings with 5% aqueous NaNCO₃ solution, followed by drying over Na₂SO₄, filtration and concentration in vacuo, afforded pure compound I (3.02 gram, 50% yield).

1-11. (canceled)

12. A method for preparing a compound of formula (I): 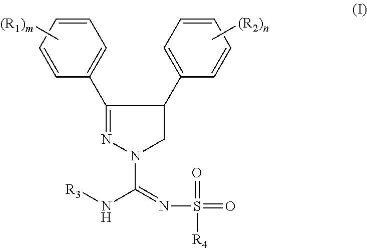
wherein:
R₁ and R₂ are independently chosen from (C₁₋₃)-alkyl, (C₁₋₃)-alkoxy, hydroxy, halogen, trifluoromethyl, trifluoromethoxy, and cyano;
m is 0, 1, or 2;
n is 0, 1, or 2;
R₃ is chosen from branched (C₁₋₈)-alkyl, linear (C₁₋₈)-alkyl, and (C₃₋₈)-cycloalkyl; and
R₄ is chosen from:
a phenyl group, a thienyl group, and a pyridyl group, wherein said group can be optionally substituted with 1 or 2 substituents independently chosen from (C₁₋₃)-alkyl, (C₁₋₃)-alkoxy, hydroxy, halogen, trifluoromethyl, trifluoromethoxy, and cyano;
a monocyclic (C₅₋₁₀)-alkyl, a bicyclic (C₅₋₁₀)-alkyl, a monocyclic (C₅₋₁₀)-alkenyl, a bicyclic (C₅₋₁₀)-alkenyl, a monocyclic hetero-(C₅₋₁₀)-alkyl, a bicyclic hetero-(C₅₋₁₀)-alkyl, a monocyclic hetero-(C₅₋₁₀)-alkenyl, and a bicyclic hetero-(C₅₋₁₀)-alkenyl; wherein said R₄ group is optionally substituted with a substituent chosen from hydroxy and (C₁₋₃)-alkyl and said monocyclic and bicyclic hetero groups contain one or two ring heteroatoms or ring heteroatom-containing moieties chosen from N, O, S and SO₂; and
a 4,4-difluoropiperidin-1-yl, a 4-fluoropiperidin-1-yl group, and
a 4-(trifluoromethyl)piperidin-1-yl group;
said process comprising the steps of:
(i) reacting a 3,4-diaryl-4,5-dihydro-(1H)-pyrazole of formula (II): 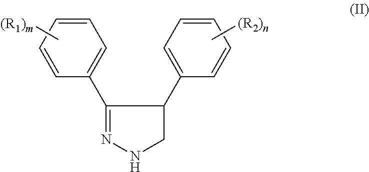
wherein R₁, R₂, m and n have the meanings given above, with an alkyl-isothiocyanate or a cycloalkylisothiocyanate of formula R₃—N═C═S, wherein R₃ has the meaning as given above, in a (C₁₋₈)-alcohol to give a 3,4-diaryl-N-alkyl-4,5-dihydro-(1H)-pyrazole-1-carbothioamide or a 3,4-diaryl-N-cycloalkyl-4,5-dihydro-(1H)-pyrazole-1-carbothioamide of formula (III): 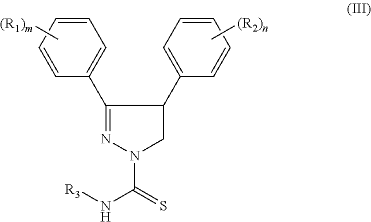
(ii) reacting the obtained compound of formula (III) with an alkylating reagent of general formula R^{x}-L, wherein Rx represents a linear (C₁₋₈)-alkyl group and L represents a ‘leaving group’, in a (C₁₋₈)-alcohol to give a compound of formula (IV): 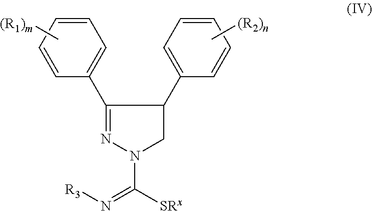
(iii) reacting the obtained compound of formula (IV), with a sulfonamide derivative of formula R₄SO₂NH₂ in an inert organic solvent to give a compound of formula (I), and
(iv) isolating the compound of formula (I) from the reaction mixture.

13. The method of claim 12, wherein
R₁ and R₂ are independently chosen from (C₁₋₃)-alkyl, trifluoromethyl, and halogen;
m is 0 or 1;
n is 0 or 1;
R₃ is selected from branched (C₁₋₃)-alkyl, and linear (C₁₋₃)-alkyl; and
R₄ is chosen from:
phenyl optionally substituted with one substituent chosen from (C₁₋₃)-alkyl, trifluoromethyl, and halogen,
a monocyclic hetero-(C₅₋₁₀)-alkyl group containing one or two ring heteroatoms chosen from N, O and S, and
a 4,4-difluoropiperidin-1-yl group, a 4-fluoropiperidin-1-yl group, and
a 4-(trifluoromethyl)piperidin-1-yl group.

14. The method of claim 12, wherein
R₁ and R₂ are halogen;
m is 0 or 1;
n is 0 or 1;
R₃ is methyl; and
R₄ is selected from phenyl optionally substituted with one halogen atom, a piperidin-1-yl group, and a 4,4-difluoropiperidin-1-yl group.

15. The method of claim 12, wherein
R₁ is 4-Cl;
m is 1;
n is 0;
R₃ is methyl; and
R₄ is chosen from a 4-chlorophenyl group, a piperidin-1-yl group, and a 4,4-difluoropiperidin-1-yl group.

16. The method of claim 12, wherein the compound of formula (I) is

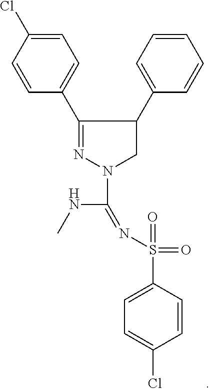

17. The method of claim 12, wherein the compound of formula (I) is

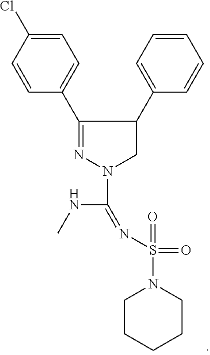

18. The method of claim 12, wherein the compound of formula (I) is

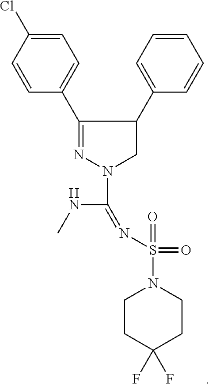

19. The method of claim 12, wherein the (C₁₋₈)-alcohol of reaction (i) is absolute ethanol, the (C₁₋₈)-alcohol of reaction (ii) is methanol, the leaving group of the alkylating reagent of reaction (ii) is chosen from Br, Cl and I, and the inert organic solvent of reaction (iii) is acetonitrile.

20. A compound of formula (III) or (IIIa): 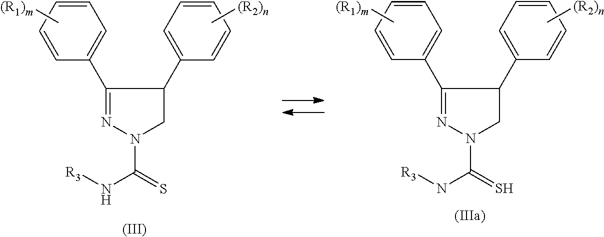
or a tautomer, stereoisomer, or N-oxide thereof, or a salt of any of the foregoing, wherein:
R₁ and R₂ independently are chosen from (C₁₋₃)-alkyl, (C₁₋₃)-alkoxy, hydroxy, halogen, trifluoromethyl, trifluoromethoxy and cyano,
m is 0, 1 or 2,
n is 0, 1, or 2, and
R₃ is chosen from branched (C₁₋₈)-alkyl and linear (C₁₋₈)-alkyl.

21. A compound of formula (IV): 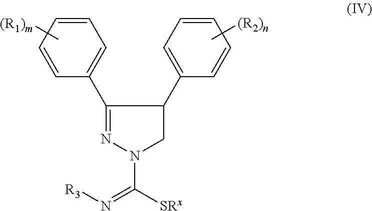
or a tautomer, stereoisomer, or N-oxide thereof, or a salt of any of the foregoing, wherein:
R₁ and R₂ independently are chosen from (C₁₋₃)-alkyl, (C₁₋₃)-alkoxy, hydroxy, halogen, trifluoromethyl, trifluoromethoxy and cyano;
m is 0, 1 or 2;
n is 0, 1, or 2;
R₃ is chosen from branched (C₁₋₈)-alkyl, linear (C₁₋₈)-alkyl, and (C₃₋₈)-cycloalkyl; and
Rx is a linear (C₁₋₈)-alkyl group.

22. The compound of formula (III) or (IIIa) as claimed in claim 20, wherein said compound is an optically active enantiomer.

23. The compound of formula (IV) as claimed in claim 21, wherein said compound is an optically active enantiomer.
